(19) United States
(12) Patent Application Publication
(10) Pub. No.: US 2025/0043044 A1
(43) Pub. Date: Feb. 6, 2025

(54) BUTADIENE PRODUCTION FROM USED TIRES

(71) Applicant: Bridgestone Americas Tire Operations, LLC, Nashville, TN (US)

(72) Inventors: Robert W. WHITE, Gilbert, AZ (US); Michael R. HARTZELL, Gold Canyon, AZ (US); William S. NIAURA, Hudson, OH (US); Mark W. SMALE, Hudson, OH (US); Terrence E. HOGAN, Uniontown, OH (US); James J. KUHEL, III, Stow, OH (US)

(21) Appl. No.: 18/718,138

(22) PCT Filed: Dec. 8, 2022
(86) PCT No.: PCT/US2022/081188

§ 371 Date: Jun. 10, 2024

Related U.S. Application Data
(60) Provisional application No. 63/329,255, filed on Apr. 8, 2022.
(60) Provisional application No. 63/287,220, filed on Dec. 8, 2021.

(51) Int. Cl. C08F 36/06; B60C 1/00; C01B 3/02; C07C 1/207; C08K 3/36; C08L 9/00; C12P 5/00

(52) U.S. Cl. CPC C08F 36/06; B60C 1/00; C01B 3/02; C07C 1/2076; C08K 3/36; C08L 9/00; C12P 5/007; C08L 2207/322

(57) ABSTRACT

A process comprising (a) providing used tire feedstock; (b) gasifying the used tire feedstock to produce a gaseous stream, where the gaseous stream includes carbon monoxide, hydrogen, and carbon dioxide; (c) biosynthetically converting at least a portion of the carbon monoxide, hydrogen, and carbon dioxide within the gaseous stream to produce a first product stream; (d) converting at least a portion of the first product stream to a second product stream, where the second product stream includes acetaldehyde and hydrogen; (e) routing a portion of the hydrogen within the second product stream to said step of biosynthetically converting at least a portion of the carbon monoxide, hydrogen, and carbon dioxide within the gaseous stream; and (f) converting at least a portion acetaldehyde to butadiene monomer.

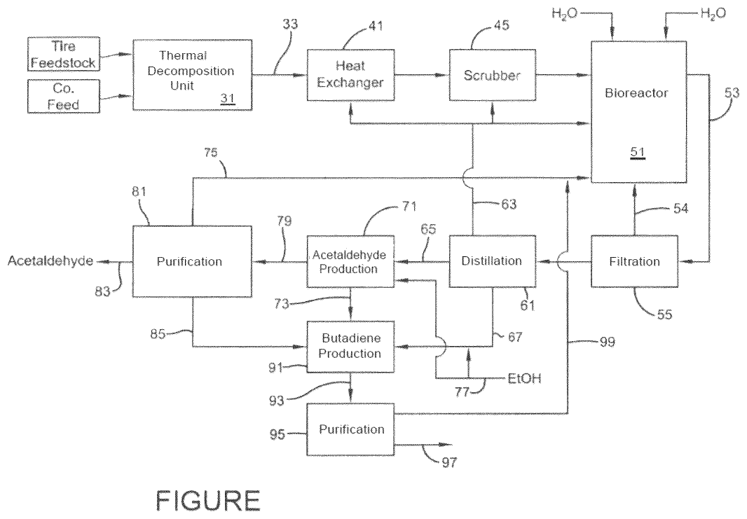

This application claims the benefit of U.S. Provisional Application Ser. No. 63/287,220 filed on Dec. 8, 2021, and U.S. Provisional Application Ser. No. 63/329,255 filed on Apr. 8, 2022, which are incorporated herein by reference

### FIELD OF THE INVENTION

Embodiments of the present invention are directed toward a process for converting used tires to butadiene monomer.

### BACKGROUND OF THE INVENTION

Butadiene monomer is polymerized to polybutadiene and butadiene copolymers such as poly(styrene-co-butadiene), poly(isoprene-co-butadiene), and poly(styrene-co-isoprene-co-butadiene). While these polymers have many uses, they are significantly used in the manufacture of tires. Used tires, on the other hand, are not easily recycled and have been landfilled or incinerated for fuel value. Methods have been proposed to thermally decompose tires into syngas, and then convert the syngas to useful materials. These methods lack industrial applicability, and therefore improvements to this general pathway are desired.

### SUMMARY OF THE INVENTION

One or more embodiments of the present invention provide a process comprising (a) providing used tire feedstock; (b) gasifying the used tire feedstock to produce a gaseous stream, where the gaseous stream includes carbon monoxide, hydrogen, and carbon dioxide; (c) biosynthetically converting at least a portion of the carbon monoxide, hydrogen, and carbon dioxide within the gaseous stream to produce a first product stream; (d) converting at least a portion of the first product stream to a second product stream, where the second product stream includes acetaldehyde and hydrogen; (e) routing a portion of the hydrogen within the second product stream to said step of biosynthetically converting at least a portion of the carbon monoxide, hydrogen, and carbon dioxide within the gaseous stream; and (f) converting at least a portion acetaldehyde to butadiene monomer.

Other embodiments of the present invention provide a process comprising (a) providing used tire feedstock; (b) optionally providing a co-feed that includes carbonaceous material other than used tire feedstock; (c) gasifying the used tire feedstock and optional co-feed to produce a gaseous stream, where the gaseous stream includes carbon monoxide, hydrogen, and carbon dioxide; (d) introducing the gaseous stream to an aqueous medium wherein the carbon monoxide, hydrogen, and carbon dioxide are converted to a first product stream; (e) converting the first product stream to a second product stream, where the second product stream includes acetaldehyde and hydrogen; (f) separating the hydrogen from the second product stream to thereby form a hydrogen stream; and (g) converting the acetaldehyde to a final product stream, where the final product stream includes butadiene.

Yet other embodiments of the present invention provide a vulcanizable composition of matter comprising the polybutadiene or butadiene copolymer prepared by either of the above processes.

Still other embodiments of the present invention provide a tire component prepared by the above vulcanizable composition.

Other embodiments of the present invention provide a tire prepared by employing the above tire component.

### BRIEF DESCRIPTION OF THE DRAWING

The FIGURE is a schematic view of a system for practicing embodiments of the invention.

### DETAILED DESCRIPTION OF ILLUSTRATIVE EMBODIMENTS

Embodiments of the invention are based, at least in part, on the discovery of a process for consuming used tires in the production of butadiene and optionally acetaldehyde. According to one or more embodiments, used tires are thermally decomposed to form a gaseous stream that is then biosynthetically converted to ethanol. The ethanol is then converted to acetaldehyde, which reaction produces a hydrogen by-product stream that is used in the upstream bio-production of ethanol. The acetaldehyde can be purified and/or converted to butadiene monomer by reacting it with ethanol. It has been discovered that the overall process efficiency and economics depends on the amount of hydrogen available during the biosynthesis of ethanol. Therefore, the present invention, which provides down-stream production of hydrogen in the absence of carbon by-products, provides for overall carbon efficiency. This is particularly advantageous in the present invention since used tires are the primary feedstock, and used tires include a higher molar ratio of carbon to hydrogen than do other feedstock such as biomass. In one or more embodiments, the butadiene is polymerized to form polybutadiene or butadiene copolymers that are used in the preparation of vulcanizable compositions that are fabricated into tire components.

### Process System and Overview

Embodiments of the invention can be described with reference to the FIGURE, which depicts system 20 for converting used tires to butadiene and optionally acetaldehyde. System 20 includes a thermal decomposition unit 31 that is followed in series by a bioreactor 51. Thermal decomposition unit 31 is in fluid communication, either directly or indirectly, with bioreactor 51 via gas stream conduit 33. An acetaldehyde synthesis unit 71, which may also be referred to as acetaldehyde production unit 71, which is downstream of bioreactor 51, is in fluid communication, either directly or indirectly, with bioreactor 51 via ethanol product conduit 53. A butadiene synthesis unit 91, which may also be referred to as butadiene production unit 91, is downstream of acetaldehyde synthesis unit 71 and is in fluid communication, either directly or indirectly, with acetaldehyde synthesis unit 71 via acetaldehyde product conduit 73. Acetaldehyde synthesis unit 71 is also in fluid communication, either directly or indirectly, with hydrogen by-product conduit 75, which is in fluid communication with bioreactor 51.

According to embodiments of the invention, thermal decomposition unit 31 is adapted to receive tire feedstock, and optionally co-feed, and thermally treat the same to produce a gaseous stream that includes carbon monoxide (CO), hydrogen gas (H₂), and optionally carbon dioxide (CO₂). Bioreactor 51 includes one or more microorganism cultures that are adapted to convert the carbon monoxide, hydrogen gas, and optionally carbon dioxide to ethanol. The ethanol is transferred to acetaldehyde synthesis unit 71 where the ethanol is converted to acetaldehyde with the byproduct production of hydrogen. The acetaldehyde can be transferred to butadiene synthesis unit 91 where the acetaldehyde is converted to butadiene. The byproduct hydrogen from acetaldehyde synthesis unit 71 can be transferred to bioreactor 51 via conduit 75 or via conduit 99, which is in fluid communication, either directly or indirectly, with butadiene synthesis unit 91.

In one or more embodiments, the gaseous stream exiting thermal decomposition unit 31 is treated prior to being introduced to bioreactor 51. For example, and as shown in the FIGURE, the gaseous product stream can be cooled within a heat exchanger 41. In one or more embodiments, heat exchanger can receive cooling water from one or more downstream processes or units such as a distillation column 61, which will be described in greater detail below. Also, the gaseous stream can be treated to remove one or more constituents prior to being introduced to bioreactor 51. For example, and as shown in the FIGURE, the gaseous stream can be treated with a scrubber 45.

In any event, carbon monoxide, hydrogen, and optionally carbon dioxide are converted to ethanol within bioreactor 51. As shown in the FIGURE, bioreactor 51 can include external inputs of hydrogen gas and water. In one or more embodiments, the ethanol produced in bioreactor 51 is transferred from bioreactor 51 within a crude product stream (e.g. the ethanol is dissolved in an aqueous media) via conduit 53. In one or more embodiments, the crude ethanol product stream can be filtered as it exits bioreactor 51 or downstream thereof by employing, for example, a filtration unit 55. As a skilled person appreciates, it is common to filter product streams exiting a bioreactor in order to prevent transfer of microorganisms to downstream processes. The skilled person also appreciates that the microorganisms, as well as carriers for the microorganisms, that are filtered from the product stream can be returned to the bioreactor. As shown in the FIGURE, microorganisms and/or carriers for the microorganisms can be returned to bioreactor 51 through microorganism-recycle conduit 57. The skilled person also understands that subsystems may be present for introducing (i.e. inoculating) bioreactor 51 with the desired microorganism culture. These subsystems may include, for example, a unit (not shown) that is in fluid communication with bioreactor 51 and is adapted to culture the microorganisms.

Prior to introducing the crude ethanol product stream to acetaldehyde synthesis unit 71, the crude ethanol stream can be concentrated or otherwise purified. For example, ethanol can be separated from the crude ethanol product stream within a distillation unit 61 where the overhead (i.e. distillate), which includes concentrated ethanol, is directed to acetaldehyde synthesis unit 71 via conduit 65 and/or butadiene production unit 91 via conduit 67, and the bottoms from the distillation can be recycled back to, for example, bioreactor 51 via aqueous bottoms conduit 63.

Ethanol is converted to acetaldehyde within acetaldehyde production unit 71, which may be referred to as acetaldehyde synthesis unit 71 or acetaldehyde reactor 71. Acetaldehyde synthesis produces a crude product stream that includes acetaldehyde and hydrogen by-byproduct. In one or more embodiments, the crude acetaldehyde product stream can be transferred, either directly or indirectly, through conduit 73, to butadiene synthesis unit 91. In other embodiments, the crude acetaldehyde is transferred, either directly or indirectly, via conduit 79, to a separation unit 81 (e.g. distillation column, which may also be referred to as purification unit 81) where the by-product hydrogen is separated from the acetaldehyde. The by-product hydrogen can be routed, either directly or indirectly, back to bioreactor 51 via conduit 75. The acetaldehyde stream from separation unit 81 can be routed to market sources via conduit 83 or to butadiene reactor 91 via conduit 85.

Acetaldehyde is converted to butadiene within synthesis unit 91 to produce a crude butadiene product stream, which can exit synthesis unit 91, directly or indirectly, via conduit 93. In one or more embodiments, butadiene is separated from the crude butadiene stream within a distillation column 95 to produce a purified butadiene stream that can be removed from the system via conduit 97. In those embodiments where crude acetaldehyde stream, which includes hydrogen, is fed to butadiene production unit 91, purification unit 95 (e.g. distillation unit 95) will produce a by-product hydrogen stream that can be routed back to bioreactor 51 via conduit 99.

As also shown in the FIGURE, acetaldehyde synthesis unit 71 and/or butadiene production unit 91 can be supplemented with an external source of ethanol via conduit 77. This external source can be from, for example, the fermentation of agricultural crops such as corn. In another embodiment, this external source can be from cellulose ethanol produced from grasses, wood, algae, or other plants.

While the system and process of the present invention is shown as a single integrated system with each unit in fluid communication, either directly or indirectly, with the other units upstream and/or downstream thereof, the skilled person will be able to readily envisage systems and methods that are less directly connected yet still integrated. For example, a system may exist whereby gasification unit 31 and bioreactor 51 are located at a first facility, and acetaldehyde production unit 71 and butadiene production unit 91 are located at a second facility. The first facility (e.g. gasification 31 and bioreactor 51) can be indirectly connected to the second facility (e.g. acetaldehyde reactor 71 and butadiene reactor 91) via, for example, a pipeline that can transport ethanol from the first facility to the second facility. Alternatively, ethanol can be transported from the first facility to the second facility via other forms of transportation including truck or railcar. Similarly, hydrogen produced at acetaldehyde reactor 71 can be communicated back to bioreactor 51 (i.e. from the second facility to the first facility) by pipeline, tanker, truck, or through exchange with local sources of hydrogen. For purposes of this specification, and unless otherwise specifically stated, indirect fluid communication will be understood to encompass these connections between the various units.

### Characteristics of Tires and Carbonaceous Material Feedstock

In one or more embodiments, the feedstock fed to thermal decomposition unit 31 includes tire feedstock from used tires, which may also be referred to as used tire feedstock. As the skilled person appreciates, tire feedstock may include vulcanized polymer, carbon black filler, silica, resins, oils, fibrous yarn, and metal. The vulcanized polymer may include the sulfur-crosslinked residue of natural rubber and/or one or more synthetic elastomers including diene polymers and copolymers. In one or more embodiments, the used tire feedstock may include shredded or otherwise ground tires with one or more constituents of the used tire removed. For example, the tire feedstock may be treated to remove metal by methods known in the art (e.g. magnetic separation). Alternatively or in combination therewith, the used tire feedstock may be optionally treated to remove fibrous reinforcement such as fiber yard or cord, which the skilled person understands is often found in conjunction with the vulcanized rubber within many tire components. Alternatively or in combination with the foregoing, the used tire feedstock may be optionally treated to remove inorganic materials such as silica filler, which the skilled person appreciates is often found used tire components. In any event, the tire feedstock can be processed into tire shreds, tire chips, or ground or crumb rubber and fed to the thermal decomposition unit.

In one or more embodiments, the tire feedstock is characterized by relatively low amounts of metal, which may result from pre-treatment of the tire feedstock to remove metal. In one or more embodiments, the tire feedstock includes less than 25 wt %, in other embodiments less than 15 wt %, and in other embodiments less than 1 wt % metal based on the entire weight of the feedstock fed to thermal decomposition in accordance with the present invention.

In one or more embodiments, the tire feedstock is characterized by relatively low amounts of fibrous yarn or cord, which may result from pre-treatment of the tire feedstock to remove fibrous yarn or cord. In one or more embodiments, the tire feedstock includes less than 5 wt %, in other embodiments less than 4 wt %, in other embodiments less than 3 wt %, in other embodiments less than 2 wt %, and in other embodiments less than 3 wt % fibrous yarn or cord based on the entire weight of the feedstock fed to thermal decomposition in accordance with the present invention.

In one or more embodiments, the tire feedstock is characterized by relatively low amounts of inorganic filler (e.g. silica), which may result from pre-treatment of the tire feedstock to remove inorganic filler. In one or more embodiments, the tire feedstock includes less than 30 wt %, in other embodiments less than 20 wt %, in other embodiments less than 10 wt %, and in other embodiments less than 5 wt % inorganic filler based on the entire weight of the feedstock fed to thermal decomposition in accordance with the present invention.

In one or more embodiments, the used tire feedstock includes tire remains from passenger tires. In other embodiments, the used tire feedstock includes tire remains from non-passenger tires such as, but not limited to, truck and bus tires, off-road vehicle tires, agricultural tires, and race tires.

In one or more embodiments, the used tires may be characterized by a compacted density of greater than 640 kg/m³, in other embodiments greater than 720 kg/m³, and in other embodiments greater than 770 kg/m³, where density is determined by ASTM D 698-07.

As indicated above, the feedstock to the thermal decomposition unit includes tire feedstock and optionally complementary feedstock. In one or more embodiments, the complementary feedstock, which may also be referred to as co-feed, includes carbonaceous materials other than the tire feed stock. Carbonaceous material refers to any carbon material whether in solid, liquid, gas, or plasma state. Non-limiting examples of carbonaceous materials include carbonaceous liquid product, industrial liquid recycle, municipal solid waste (MSW or msw), urban waste, agricultural material, forestry material, wood waste, construction material, vegetative material, industrial waste, fermentation waste, petrochemical coproducts, alcohol production coproducts, coal, plastics, waste plastic, coke oven tar, lignin, black liquor, polymers, waste polymers, polyethylene terephthalate (PETA), polystyrene (PS), sewage sludge, animal waste, crop residues, energy crops, forest processing residues, wood processing residues, livestock wastes, poultry wastes, food processing residues, ethanol coproducts, spent grain, spent microorganisms, municipal waste, construction waste, demolition waste, biomedical waste, hazardous waste, or their combinations. In one or more embodiments, the carbonaceous material includes biomass. In one or more embodiments, the biomass is bagasse including, but not limited to, the bagasse of sugar cane, sorghum, and guayule plant.

In a further embodiment, the guayule bagasse is produced as the result of a process to extract rubber and resin from the guayule plant, such as described in U.S. Publication No. 2022/0356273 A1, which is incorporated herein by reference. Methods for the desolventization of guayule bagasse are described in U.S. Pat. No. 10,132,563, which is also incorporated herein by reference. In one or more embodiments, the guayule bagasse contains no more than 1 wt % organic solvent (based upon the total weight of the dried bagasse). In certain embodiments, the dried bagasse contains no more than 0.5 wt % organic solvent (based upon the total weight of the dried bagasse). In one or more embodiments, the dried bagasse may contain a quantity of water and higher boiling point terpenes. In certain embodiments, the total quantity of water and higher boiling point terpenes in the dried bagasse may be higher than the content of organic solvents. In certain embodiments, resin content (including the higher boiling point terpenes) in the dried bagasse is generally acceptable and in some instances actually preferred.

In one or more embodiments, the co-feed (e.g. biomass or municipal waste) may be characterized by a compacted density of less than 600 kg/m³, in other embodiments less than 580 kg/m³, and in other embodiments less than 560 kg/m³, where density is determined by ASTM D 698-07.

The feedstock may be characterized by the amount of co-feed (e.g. biomass or municipal waste). In one or more embodiments, the feedstock includes from about 0_to about 95, in other embodiments from about 1 to about 75, and in other embodiments from about 2 to about 55 wt % co-feed with the balance including used tire. In one or more embodiments, the feedstock includes less than 95, in other embodiments less than 80, and in other embodiments less than 70 wt % co-feed. In these or other embodiments, the feedstock includes greater than 10, in other embodiments greater than 20, in other embodiments greater than 30, in other embodiments greater than 40, in other embodiments greater than 50, and in other embodiments greater than 70 wt % used tires, with the balance including complementary feedstock.

### Thermal Decomposition of Tires and Optionally Biomass

According to embodiments of the invention, the feedstock (which includes tire feedstock and optionally co-feed) are thermally decomposed into gaseous streams including hydrogen, carbon monoxide, and optionally carbon dioxide by employing techniques that are generally known in the art. As the skilled person understands, these processes may include gasification processes, and it is also known that these processes can be tailored to control the chemical nature of the resulting gaseous stream. For example, the degree of combustion can be controlled by controlling the amount of oxygen present during thermal decomposition. In one or more embodiments, the step of thermal decomposition takes place in a substantially inert environment.

Processes that may be used for the thermal decomposition step may include pyrolysis reactions as disclosed in U.S. Publication Nos. 20210207037; 20190295734; 20190249089; 20180273415; 20170009162; 20170002271; 20160107913; 20160068773; 20160024404; 20140182205; 20140157667; and 20140100294, which are incorporated herein by reference.

In one or more embodiments, where the feedstock includes both tire feedstock and co-feed, the tire feedstock and the co-feed can be introduced to the same thermal decomposition unit simultaneously. For example, the tire feedstock and the co-feed can be pre-mixed at a desired ratio to form the feedstock that is fed to the thermal decomposition unit. Alternatively, separate streams of tire feedstock and co-feed can be separately and individually fed to the thermal decomposition unit at a desired rate. In yet other embodiment, the two feedstocks (i.e. the tire feedstock and the co-feed) can be sequentially treated within the same thermal decomposition unit. In still other embodiments, the two feedstocks (i.e. the tire feedstock and the co-feed) can be treated within separate thermal decomposition units operating in parallel, and the then the gaseous streams produced by the respective units can be combined to attain the desired ratio of gaseous constituents.

### Characteristics of Gaseous Product Stream

As indicated above, the gaseous product stream produced by thermal decomposition unit 31 includes carbon monoxide, hydrogen and optionally carbon dioxide. In one or more embodiments, the gaseous product stream includes from about 5 to about 50, or in other embodiments from about 7 to about 25, or in other embodiments from about 8 to about 15 volume percent carbon dioxide. In these or other embodiments, the gaseous product stream includes from about 10 to about 85, or in other embodiments from about 20 to about 65, or in other embodiments from about 25 to about 45 volume percent hydrogen. In these or other embodiments, the gaseous product stream includes from about 20 to about 85, or in other embodiments from about 30 to about 75, or in other embodiments from about 40 to about 60 volume percent carbon monoxide. In one or more embodiments, the gaseous product stream produced by thermal decomposition includes from about 40 to about 80 wt %, in other embodiments from about 45 to about 75 wt %, and in other embodiments from about 50 to about 70 wt % carbon (i.e. carbon within carbon-based compounds) based on the total weight of the gaseous product stream.

### Conditioning of Gaseous Stream

In one or more embodiments, the gaseous stream is conditioned (i.e. treated) prior to providing the stream to the bioreactor 21. In one or more embodiments, the gaseous product stream from thermal decomposition, which is carried by conduit 33, may be pressurized. In one or more embodiments, pressurization of the gaseous stream achieves sufficient pressure to overcome counter forces within the bioreactor. As the skilled person understands, this will permit flow of the gas through the bioreactor and allow inert gases (e.g. nitrogen) within the gaseous stream to enter the head space of the reactor. In one or more embodiments, the gaseous stream is pressurized to a pressure of from about 5 to about 20 barr.

Also, the gaseous stream can be cooled at heat exchanger 41. As the skilled person will appreciate, heat exchanger 41 may include a water-cooled unit. In one or more embodiments, the gaseous stream is cooled to a temperature below that which would otherwise have a deleterious impact on the microorganism culture within the bioreactor. In one or more embodiments, the gaseous stream is cooled to a temperature of from about 25 to about 45° C. prior to delivery to the bioreactor.

Still further, the gaseous stream can be treated with scrubber 45 prior to being introduced to the bioreactor. In one or more embodiments, this may include the use of a catalyst to remove hydrogen sulfide (e.g. iron oxide). The stream may also be treated to remove halides (e.g. treatment with calcium or sodium carbonate).

### Bioreactor

As indicated above, bioreactor 51 includes one or more microorganisms that consume one or more constituents of the gaseous product stream and produce ethanol. In one or more embodiments, bioreactor 51 may include a single reaction vessel or it may include a plurality (i.e. two or more) of reaction vessels that may operate in a complementary fashion. For example, the two or more reactor vessels may operate in parallel or in series to facilitate the desired reaction (i.e. bioconversion of the gaseous product stream to ethanol). The skilled person generally appreciates the appropriate conditions that should be maintained with the bioreactor to sustain the microorganisms and promote the desired reaction. In one or more embodiments, water is both a reactant and serves as the reaction medium within the bioreactor.

In one or more embodiments, the reactor medium within the bioreactor is maintained at a temperature of from about 30 to about 45° C. In these or other embodiments, the reaction medium within the bioreactor is maintained at a pH of from about 4 to about 7.

In one or more embodiments, the bioreactor includes at least one inlet for the introduction of the gaseous stream into the bioreactor and at least one outlet for removing a product stream from the bioreactor. In one or more embodiments, the bioreactor includes an outlet for gaseous by-product stream. In one or more embodiments, the bioreactor is a closed system but for the inlets and outlets. In other embodiments, the bioreactor is an open system. In one or more embodiments, the bioreactor is selected from a continuous stirred tank reactor, a gas lift reactor, a loop reactor, and fluidized bed reactor. In one or more embodiments, the bioreactor has a capacity of greater than 500 L, in other embodiments greater than 1000 L, and in other embodiments greater than 1500 L.

### Microorganisms

Microorganisms, or genetically-modified microorganisms, that are capable of, or adapted to, synthesize ethanol from the gaseous product stream are generally known in the art. For example, Zymomonas mobilis or Lactococcus strains, as well as certain Clostridium strains are known to produce ethanol from carbon-containing gaseous substrates. The art is replete with other useful examples as shown in U.S. Publication Nos. 20210284592; 20200255362; 20200156973; 20180264375; 20170226538; 20170225098; 20170183690; 20160160223; 20160017276; 20160010116; 20150376654; 20150353965; 20150337341; 20150299737; 20150152441; 20150087037; 20140377826; 20130316424; 20130252230; 20130230894; and 20130224839, which are incorporated herein by reference.

Ethanol-Containing Product Stream from Bioreactor

As indicated above, ethanol exits bioreactor 51 within an aqueous product stream, which may be referred to as the ethanol product stream. This aqueous product stream can be filtered as the stream exits the bioreactor. During operation, filtering of the product stream as the stream leaves the bioreactor can prevent transfer of any media that is used to immobilize the microorganisms and thereby help prevent transfer of the microorganisms from the bioreactor to downstream processes. In addition to or in lieu of filtering the ethanol-containing product stream as the stream leaves the bioreactor, the product stream can be filtered and/or sterilized at one or more intermediate units positioned downstream of the bioreactor. In addition to or in lieu of a filtration unit, the product stream may undergo separation within, for example, a centrifugation unit. Or, in other embodiments, in addition to or in lieu of filtration or centrifugation, a clarification unit (e.g. a settling tank) may be employed to further treat the product stream. In lieu of or in addition to filtration, centrifugation, and/or clarification, the product stream may undergo sterilization. For example, a sterilization unit may take advantage of UV sterilization, heat, or gamma radiation to treat the stream.

### Ethanol Concentration/Separation

As indicated above, following optional treatment at unit 55, the ethanol-containing product stream can be treated to separate ethanol from the other constituents of the aqueous stream. This may include distilling the ethanol-containing stream within separation unit 61. According to these embodiments, ethanol can be collected as an overhead stream that may be characterized by an ethanol concentration of greater than 80 wt %, in other embodiments greater than 90 wt %, and in other embodiments greater than 93 wt %. In these or other embodiments, the overhead stream (i.e. the ethanol-containing stream) may include less than 10 wt %, in other embodiments less than 8 wt %, and in other embodiments less than 1 wt % water.

In one or more embodiments, the overhead ethanol stream can optionally be further treated to purify the ethanol stream prior to introducing the ethanol to acetaldehyde production unit 71. For example, the ethanol stream can be dehydrated or dried by treating the stream in one or more water adsorption beds that include a drying material such as molecular sieves.

### Acetaldehyde Production

As indicted above, ethanol is converted to acetaldehyde within production unit 71. In one or more embodiments, substantially all of the ethanol produced in bioreactor 51 (i.e. substantially all of the ethanol within the ethanol-containing product stream) is introduced to acetaldehyde production unit 71. In these or other embodiments, ethanol obtained from outside of the process of the present invention (e.g. ethanol from fermentation of agricultural crops) is also introduced to acetaldehyde production unit 71 to supplement production of the acetaldehyde. In one or more embodiments, the weight ratio of ethanol supplied to acetaldehyde production unit 71 from bioreactor 51 to the ethanol supplied to acetaldehyde production unit 71 from other sources (e.g. ethanol from fermentation of agricultural crops) is from about 1:0 to about 1:10, in other embodiments from about 1:0.3 to about 1:7, and in other embodiments from about 1:1 to about 1:5.

As the skilled person appreciates, synthesis of acetaldehyde involves the partial dehydrogenation of ethanol to yield a hydrogen by-product stream. As described above, the hydrogen can be routed to bioreactor 51, which can advantageously offset the hydrogen deficiencies of the process. Those skilled in the art will appreciate that the introduction of ethanol from sources outside of the bioreactor process (i.e. outside of bioreactor 51) will further alleviate hydrogen deficiencies within bioreactor 51 if the additional ethanol is converted to acetaldehyde, which will allow for greater production of hydrogen.

In one or more embodiments, within acetaldehyde production unit 71, the ethanol undergoes dehydrogenation at elevated temperatures over an appropriate catalyst, such as a copper-based catalyst. For example, the reaction can take place within a fixed-bed reactor. In one or more embodiments, dehydrogenation of ethanol within unit 71 takes place at a temperature of from about 200 to about 350° C., or in other embodiments from about 250 to about 300.

### Butadiene Production

In one or more embodiments, acetaldehyde produced in unit 71 is converted to butadiene monomer within butadiene production unit 91. In one or more embodiments, butadiene production includes reacting ethanol and acetaldehyde to produce 1,3-butadiene by utilizing reaction techniques generally known in the art as for example described by Zhang, Mechanistic Insight into the Meerwein-Ponndorf-Verley Reaction and Relative Side Reactions over MgO in the Process of Ethanol to 1,3-butadiene: a DFT Study, IND. ENG. CHEM. RES., 2021, 60, 2871-2880. As the skilled person appreciates, this reaction can be conducted over an appropriate catalyst, such as a tantalum-promoted porous silica catalyst, at elevated temperatures. Also, other catalysts for converting the acetaldehyde and ethanol to butadiene are known in the art and can be used. In one or more embodiments, this reaction is conducted within a fixed-bed reactor operating at temperatures of from about at 300 to about 450° C., or in other embodiments, or in other embodiments from about 350 to about 400.

In one or more embodiments, the reactant feed into butadiene production unit 91 includes an ethanol to acetaldehyde molar ratio (i.e. moles of ethanol to moles of acetaldehyde) of at least 1:1, in other embodiments at least 2:1, in other embodiments at least 2.5:1, in other embodiments at least 4:1, and in other embodiments within the range of from about 1:1 to about 5:1.

The feed into butadiene production unit 91 is characterized by low levels of impurities (i.e. constituents other than acetaldehyde and ethanol). In one or more embodiments, the feed stream into butadiene production unit 91 includes less than 10, in other embodiments less than 5, and in other embodiments less than 2 wt % impurities based on the total weight of the input stream.

The crude butadiene stream exiting butadiene reactor 91 via conduit 93 generally includes 1,3-butadiene monomer, unreacted ethanol, unreacted acetaldehyde, water, which is a by-product of the reaction, and other side products. In one or more embodiments, yield of 1,3-butadiene, based upon acetaldehyde, is greater than 20 mol %, in other embodiments greater than 30 mol %, and in other embodiments greater than 40 mol %. In these or other embodiments, yield of 1,3-butadiene, based upon acetaldehyde, is less than 70 mol %, in other embodiments less than 60 mol %, and in other embodiments less than 55 mol %.

In one or more embodiments, the crude butadiene product stream undergoes a first separation, which may include a distillation. In one or more embodiments, butadiene is separated as an overhead stream and the remaining constituents of the stream are separated as a bottoms stream. The bottoms stream can then undergo further separation to separate the ethanol and acetaldehyde from the water and other constituents of the stream. The ethanol and acetaldehyde, which can be separated as an overhead stream, can then be recycled back to butadiene production unit 91 for conversion to butadiene.

### INDUSTRIAL APPLICABILITY

In one or more embodiments, the 1,3-butadiene produced by the methods of this invention can be used in the production of polybutadiene or butadiene copolymers (which may also be referred to as polybutadiene copolymers) that can be used in the manufacture of tire components. For purposes of this specification, these polymers may be referred to as circular synthetic rubber, or circular synthetic polybutadiene to butadiene copolymers. As a result, practice of the present invention provides a method by which waste material from used tires is converted back to useful tires. In other words, a tire recycling method is provided.

The synthesis of polybutadiene or polybutadiene copolymers from butadiene monomer is well known and can be accomplished by using several synthetic routes (i.e. polymerization mechanisms and techniques). For example, the monomer can be polymerized by free-radical emulsion polymerization, anionic polymerization, or coordination catalysis using, for example, nickel or neodymium-based catalyst systems.

As the skilled person appreciates, comonomers that can be copolymerized with butadiene to form polybutadiene copolymers include, but are not limited to, vinyl aromatic monomer such as styrene, as well as other diene monomer such as isoprene. Such other monomer(s) can be derived from sustainable processes. The polymers synthesized from butadiene produced by embodiments of the invention may be referred to as vulcanizable polymers, or as elastomeric polymers, and generally include polydienes and polydiene copolymers. Specific of polymers that can be produced and used in the manufacture of tires include, but are not limited to, polybutadiene, poly(styrene-co-butadiene), poly(styrene-co-isoprene-co-butadiene), poly(isoprene-co-butadiene), and functionalized derivatives thereof.

The polybutadiene and polybutadiene copolymers produced by the present invention exhibit excellent viscoelastic properties and are particularly useful in the manufacture of various tire components including, but not limited to, tire treads, sidewalls, subtreads, and bead fillers. These polymers can be used as all or part of the elastomeric component of a tire stock. When the polymers produced by the present invention are used in conjunction with other vulcanizable polymers to form the elastomeric component of a tire stock, these other vulcanizable polymers may include natural rubber, synthetic rubbers, and mixtures thereof. Examples of synthetic rubber include polyisoprene, poly(styrene-co-butadiene), and other polybutadienes with low and/or cis-1,4-linkage content, poly(styrene-co-butadiene-co-isoprene), and mixtures thereof. The polymers of this invention can also be used in the manufacture of hoses, belts, shoe soles, window seals, other seals, vibration damping rubber, and other industrial products.

Practice of the present invention not only offers a method for recycling tires by employing used tires as a feed stock to produce polymer that can be formulated back into tires, but the practice of the present invention also advantageously provides a method whereby a tire is produced that has a relatively high content of sustainable constituents, which include recycled materials or naturally-derived materials. Moreover, these tires or tire components include threshold amounts of circular synthetic rubber while being characterized by high sustainable content. For example, the tires or tire components of the present invention can include greater than 40 wt %, in other embodiments greater than 50 wt %, and in other embodiments greater than 60 wt % sustainable materials. In these or other embodiments, the tire or tire components include from about 40 to about 90 wt %, in other embodiments from about 45 to about 85 wt %, and in other embodiments from about 50 to about 80 wt % sustainable material. In combination therewith, the rubber component of the tires or tire components of the present invention include greater than 10 wt %, in other embodiments greater than 20 wt %, in other embodiments greater than 30 wt %, in other embodiments greater than 40 wt %, in other embodiments greater than 45 wt %, and in other embodiments greater than 50 wt % circular synthetic rubber, which includes synthetic rubber produced according to embodiments of the present invention.

As indicated above, the vulcanizable compositions of this invention include a rubber component. This rubber component includes the circular synthetic rubber produced according to aspects of this invention. The rubber component may also include other synthetic rubber, such as synthetic rubber that derives from petroleum-based raw materials and has not been recycled, synthetic rubber that derives from other sustainable processes, as well as natural rubber. As the skilled person understands, natural rubber is synthesized by and obtained from plant life. For example, natural rubber can be obtained from Hevea rubber trees, guayule shrub, gopher plant, mariola, rabbitbrush, milkweeds, goldenrods, pale Indian plantain, rubber vine, Russian dandelions, mountain mint, American germander, and tall bellflower.

Other synthetic polymers, if used, can include, without limitation, synthetic polyisoprene, polybutadiene, polyisobutylene-co-isoprene, neoprene, poly(ethylene-co-propylene), poly(styrene-co-butadiene), poly(styrene-co-isoprene), poly(styrene-co-isoprene-co-butadiene), poly(isoprene-co-butadiene), poly(ethylene-co-propylene-co-diene), polysulfide rubber, acrylic rubber, urethane rubber, silicone rubber, epichlorohydrin rubber, and mixtures thereof. These elastomers can have a myriad of macromolecular structures including linear, branched, and star-shaped structures.

Generally, the rubber compositions of this invention include from about 30 to about 65 wt %, in other embodiments from about 35 to about 60 wt %, and in other embodiments from about 40 to about 55 wt % elastomer, based on the total weight of the tire component.

As suggested above, the rubber compositions include fillers such as organic and inorganic fillers. Examples of organic fillers include carbon black and starch. Examples of inorganic fillers include silica, aluminum hydroxide, magnesium hydroxide, mica, talc (hydrated magnesium silicate), and clays (hydrated aluminum silicates). In certain embodiments, a mixture of different fillers may be advantageously employed.

The amount of total filler employed in the rubber compositions can be up to about 150 parts by weight per 100 parts by weight of rubber (phr), with about 30 to about 125 phr, or about 40 to about 110 phr being typical. In certain embodiments the total filler content is greater than about 100 phr. In other embodiments, the total filler content is from about 50 to about 100 phr, and in in further embodiments from about 55 to about 95 phr.

Conventional carbon black can be used, which is generally known in the art. In one or more embodiments, carbon blacks include furnace blacks, channel blacks, and lamp blacks. More specific examples of carbon blacks include super abrasion furnace blacks, intermediate super abrasion furnace blacks, high abrasion furnace blacks, fast extrusion furnace blacks, fine furnace blacks, semi-reinforcing furnace blacks, medium processing channel blacks, hard processing channel blacks, conducting channel blacks, and acetylene blacks.

In particular embodiments, the carbon blacks may have a surface area (EMSA) of at least 20 m²/g and in other embodiments at least 35 m²/g; surface area values can be determined by ASTM D-1765 using the cetyltrimethylammonium bromide (CTAB) technique. The carbon blacks may be in a pelletized form or an unpelletized flocculent form. The preferred form of carbon black may depend upon the type of mixing equipment used to mix the rubber compound.

In one or more embodiments, carbon black can be sourced from a recycled material. Such recycled material can include reclaimed or recycled vulcanized rubber, whereby the vulcanized rubber is typically reclaimed from manufactured articles such as a pneumatic tire, an industrial conveyor belt, a power transmission belt, and a rubber hose. The recycled carbon black may be obtained by a pyrolysis process or other methods known for obtaining recycled carbon black. In an aspect, a recycled carbon black can be formed from incomplete combustion of recycled rubber feedstock or rubber articles. In another aspect, the recycled carbon black can be formed from the incomplete combustion of feedstock including oil resulting from the tire pyrolysis process. The carbon blacks utilized in the preparation of the vulcanizable elastomeric compositions can be in pelletized form or an unpelletized flocculent mass.

The amount of carbon black employed in the rubber compositions can be up to about 75 parts by weight per 100 parts by weight of rubber (phr), with about 5 to about 60 phr, or about 10 to about 55 phr being typical.

The rubber composition can further include filler in the form of one or more recycled rubbers in a particulate form. Recycled particulate rubber is typically broken down and reclaimed (or recycled) by any of a plurality of processes, which can include physical breakdown, grinding, chemical breakdown, devulcanization, cryogenic grinding, a combination thereof, etc. The term recycled particulate rubber can relate to both vulcanized and devulcanized rubber, where devulcanized recycle or recycled rubber (reclaim rubber) relates to rubber which has been vulcanized, ground into particulates and may have further undergone substantial or partial devulcanization. In an example, the recycled particulate rubber used in the rubber composition is essentially free of recycled rubber resulting from devulcanization. In a situation where the vulcanized rubber contains wire or textile fiber reinforcement, such wire or fiber reinforcement can be removed by any suitable process such as magnetic separation, air aspiration and/or air flotation step. In certain embodiments, the “recycled particulate rubber” comprises cured, i.e., vulcanized (crosslinked) rubber that has been ground or pulverized into particulate matter having a mean average particle size as discussed below.

Certain silicas may be considered sustainable materials. Some commercially available silicas which may be used as sustainable materials for the current invention include Hi-Sil™ 215, Hi-Sil™ 233, and Hi-Sil™ 190 (PPG Industries, Inc.; Pittsburgh, Pa.). Other suppliers of commercially available silica include Grace Davison (Baltimore, Md.), Degussa Corp. (Parsippany, N.J.), Rhodia Silica Systems (Cranbury, N.J.), and J.M. Huber Corp. (Edison, N.J.). Other sustainable silicas include those derived from rice husk ash.

In one or more embodiments, silicas may be characterized by their surface areas, which give a measure of their reinforcing character. The Brunauer, Emmet and Teller (“BET”) method (described in J. Am. Chem. Soc., 1939, vol. 60, 2 p. 309-319) is a recognized method for determining the surface area. The BET surface area of silica is generally less than 450 m²/g. Useful ranges of surface area include from about 32 to about 400 m²/g, about 100 to about 250 m²/g, and about 130 to about 240 m²/g, and about 170 to about 220 m²/g. In certain embodiments, the silica may have a BET surface area of 190 to about 280 m²/g. The pH's of the silicas are generally from about 5 to about 7 or slightly over 7, or in other embodiments from about 5.5 to about 6.8.

In one or more embodiments, where silica is employed as a filler (alone or in combination with other fillers), a coupling agent and/or a shielding agent may be added to the rubber compositions during mixing in order to enhance the interaction of silica with the elastomers. Useful coupling agents and shielding agents are disclosed in U.S. Pat. Nos. 3,842,111; 3,873,489; 3,978,103; 3,997,581; 4,002,594; 5,580,919; 5,583,245; 5,663,396; 5,674,932; 5,684,171; 5,684,172; 5,696,197; 6,608,145; 6,667,362; 6,579,949; 6,590,017; 6,525,118; 6,342,552; and 6,683,135; which are incorporated herein by reference.

The amount of silica employed in the rubber compositions can be from about 1 to about 150 phr or in other embodiments from about 5 to about 130 phr. The useful upper range is limited by the high viscosity imparted by silicas. In certain embodiments, the silica employed in the rubber composition is derived from rice husk ash only, and in other embodiments the rubber compositions do not include silica from non-rice husk ash derived processes. When silica is used together with carbon black, the amount of the silica or carbon black individually can be as low as about 1 phr. Generally, the amounts of coupling agents and shielding agents range from about 4 wt % to about 20 wt % based on the weight of silica used. In one or more embodiments, where carbon black and silica are employed in combination as a filler, the weight ratio or silica to total filler may be from about 5 wt % to about 99 wt % of the total filler, in other embodiments from about 10 wt % to about 90 wt % of the total filler, or in yet other embodiments from about 50 wt % to about 85 wt % of the total filler. In certain embodiments the silica and carbon black fillers employed in the rubber composition are selected from the group consisting of sustainable pyrolysis carbon black and/or rice husk ash derived silica.

A multitude of rubber curing agents (also called vulcanizing agents) may be employed, including sulfur or peroxide-based curing systems. Curing agents are described in Kirk-Othmer, ENCYCLOPEDIA OF CHEMICAL TECHNOLOGY, Vol. 20, pgs. 365-468, (3^{rd} Ed. 1982), particularly Vulcanization Agents and Auxiliary Materials, pgs. 390-402, and A. Y. Coran, Vulcanization, ENCYCLOPEDIA OF POLYMER SCIENCE AND ENGINEERING, (2^{nd} Ed. 1989), which are incorporated herein by reference. Vulcanizing agents may be used alone or in combination.

Other ingredients that are typically employed in rubber compounding may also be added to the rubber compositions. These include accelerators, accelerator activators, oils, plasticizer, waxes, scorch inhibiting agents, processing aids, zinc oxide, tackifying resins, reinforcing resins, fatty acids such as stearic acid, peptizers, and antidegradants such as antioxidants and antiozonants.

With regard to oils, sustainable oils, which include plant-based oils and bio-based oils, may be used. Plant-based oils may include plant-based triglycerides. Exemplary oils include, without limitation, palm oil, soybean oil (also referred to herein as soy oil), rapeseed oil, sunflower seed, peanut oil, cottonseed oil, oil produced from palm kernel, coconut oil, olive oil, corn oil, grape seed oil, hemp oil, linseed oil, rice oil, safflower oil, sesame oil, mustard oil, flax oil. Other examples include nut-derived oils such oils obtained from beech nuts, cashews, mongongo nuts, macadamia nuts, pine nuts, hazelnuts, chestnuts, acorns, almonds, pecans, pistachios, walnuts, or brazil nuts. As the skilled person will appreciate, these oils can be produced by any suitable process such as mechanical extraction (e.g., using an oil mill), chemical extraction (e.g., using a solvent, such as hexane or carbon dioxide), pressure extraction, distillation, leaching, maceration, purification, refining, hydrogenation, sparging, etc.

Bio-based oils, also referred to as bio-oils, can include oils produced by a recombinant cell. For example, bio-oils produced by recombinant cells can be produced using a select strain of algal cells that are fed with a supply of sugars (e.g., sucrose) and then allowed to ferment and produce a bio-oil with a selected profile; after sufficient growth or fermentation has taken place, the bio-oil is isolated from the cells and collected.

Generally, the rubber compositions of this invention can include from about 1 to about 70 parts by weight, or in other embodiments from about 5 to about 50 parts weight total oil per 100 parts by weight rubber. The amount of sustainable oil, relative to the total weight of oil included, may be from about 1 wt % to about 99 wt %, or in other embodiment from about 20 wt % to about 80 wt %.

With regard to waxes, the rubber compositions can include one or more sustainable waxes, which include natural waxes. A natural wax, or one with no petroleum as its raw material, can include carnauba wax, candelilla wax (e.g., extracted from candelilla flowers), rice wax (e.g., separated from rice bran oil) and Japan wax (e.g., extracted from Japanese wax tree).

Generally, the rubber compositions of this invention include from about 1 to about 20 parts by weight, or in other embodiments from about 2 to about 15 parts by weight total wax per 100 parts by weight rubber. The amount of sustainable wax, relative to the total weight of wax included, may be from about 1 wt % to about 99 wt %, or in other embodiment from about 20 wt % to about 80 wt % of the total wax. In certain embodiments, the rubber composition includes sustainable waxes only.

All ingredients of the rubber compositions can be mixed with standard mixing equipment such as Banbury or Brabender mixers, extruders, kneaders, and two-rolled mills. In one or more embodiments, the ingredients are mixed in two or more stages. In the first stage (often referred to as the masterbatch mixing stage), a so-called masterbatch, which typically includes the rubber component and filler, is prepared. To prevent premature vulcanization (also known as scorch), the masterbatch may exclude vulcanizing agents. The masterbatch may be mixed at a starting temperature of from about 25° C. to about 125° C. with a discharge temperature of about 135° C. to about 180° C. Once the masterbatch is prepared, the vulcanizing agents may be introduced and mixed into the masterbatch in a final mixing stage, which is typically conducted at relatively low temperatures so as to reduce the chances of premature vulcanization. Optionally, additional mixing stages, sometimes called remills, can be employed between the masterbatch mixing stage and the final mixing stage. One or more remill stages are often employed where the rubber composition includes silica as the filler. Various ingredients including the polymers of this invention can be added during these remills.

The mixing procedures and conditions particularly applicable to silica-filled tire formulations are described in U.S. Pat. Nos. 5,227,425; 5,719,207; and 5,717,022, as well as European Patent No. 890,606, all of which are incorporated herein by reference. In one embodiment, the initial masterbatch is prepared by including the polymer and silica in the substantial absence of coupling agents and shielding agents.

In order to fabricate tire components with the polymers produced by the invention, the skilled person appreciates that the polymers are mixed with various other ingredients (e.g. filler and curative) to produce a rubber composition (also referred to as vulcanizable composition) that can be processed into tire components according to ordinary tire manufacturing techniques including standard rubber shaping, molding and curing techniques. Typically, vulcanization is effected by heating the vulcanizable composition in a mold; e.g., it may be heated to about 140° C. to about 180° C. Cured or crosslinked rubber compositions may be referred to as vulcanizates, which generally contain three-dimensional polymeric networks that are thermoset. The other ingredients, such as fillers and processing aids, may be evenly dispersed throughout the crosslinked network. Pneumatic tires can be made as discussed in U.S. Pat. Nos. 5,866,171; 5,876,527; 5,931,211; and 5,971,046, which are incorporated herein by reference.

In one or more embodiments, the tires can be constructed by using non-petroleum materials in place of synthetic fibers, for example, mechanical recycled fibers, chemical recycled fibers, or bio-based fibers. Likewise, the tires can be constructed by using recycled metals.

Various modifications and alterations that do not depart from the scope and spirit of this invention will become apparent to those skilled in the art. This invention is not to be duly limited to the illustrative embodiments set forth herein.

1. A process comprising:
(a) providing used tire feedstock;
(b) gasifying the used tire feedstock to produce a gaseous stream, where the gaseous stream includes carbon monoxide, hydrogen, and carbon dioxide;
(c) biosynthetically converting at least a portion of the carbon monoxide, hydrogen, and carbon dioxide within the gaseous stream to produce a first product stream;
(d) converting at least a portion of the first product stream to a second product stream, where the second product stream includes acetaldehyde and hydrogen;
(e) routing a portion of the hydrogen within the second product stream to said step of biosynthetically converting at least a portion of the carbon monoxide, hydrogen, and carbon dioxide within the gaseous stream; and
(f) converting at least a portion acetaldehyde to butadiene monomer.

2. The process of claim 1, where the first product stream includes ethanol.

3. The process of claim 1, further comprising the steps of polymerizing the butadiene monomer to polybutadiene or a polybutadiene copolymer, and fabricating a tire component with the polybutadiene or polybutadiene copolymer.

4. (canceled)

5. The process of claim 1, where said step of gasifying includes gasifying used tire feedstock and a co-feed that includes carbonaceous material other than used tire feedstock.

6. A process comprising:
(a) providing used tire feedstock;
(b) optionally providing a co-feed that includes carbonaceous material other than used tire feedstock;
(c) gasifying the used tire feedstock and optional co-feed to produce a gaseous stream, where the gaseous stream includes carbon monoxide, hydrogen, and carbon dioxide;
(d) introducing the gaseous stream to an aqueous medium wherein the carbon monoxide, hydrogen, and carbon dioxide are converted to a first product stream;
(e) converting the first product stream to a second product stream, where the second product stream includes acetaldehyde and hydrogen;
(f) separating the hydrogen from the second product stream to thereby form a hydrogen stream; and
(g) converting the acetaldehyde to a final product stream, where the final product stream includes butadiene.

7. The process of claim 6, where the first product stream includes ethanol, and where the ethanol is converted to the second product stream.

8. (canceled)

9. (canceled)

10. (canceled)

11. (canceled)

12. (canceled)

13. (canceled)

14. The process of claim 6, where said step of introducing the gaseous stream to the aqueous medium takes place within a bioreactor that contains one or more microorganisms for converting the carbon monoxide, hydrogen, and carbon dioxide to the first product stream.

15. (canceled)

16. (canceled)

17. (canceled)

18. (canceled)

19. (canceled)

20. The process of claim 6, where the hydrogen stream is introduced to the aqueous medium.

21. The process of claim 6, further comprising the step of introducing a second hydrogen stream to the aqueous medium.

22. The process of claim 7, where the step of converting the ethanol to a second product stream takes place within an acetaldehyde reactor, and further comprising the step of introducing a second stream of ethanol from external sources to the acetaldehyde reactor.

23. (canceled)

24. The process of claim 6, further comprising converting the butadiene to a polybutadiene or butadiene copolymer.

25. The process of claim 24, further comprising producing a tire material from the polybutadiene or butadiene copolymer.

26. The process of claim 6, where said step of gasifying includes gasifying the tire feedstock and co-feed.

27. The process of claim 6, where the co-feed includes biomass.

28. The process of claim 27, where the biomass includes bagasse.

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. A tire component prepared from a vulcanizable composition comprising the polybutadiene or butadiene copolymer of claim 24.

48. (canceled)

49. The tire component of claim 47, where the tire includes greater than 40 wt % sustainable material.

50. The tire component of claim 49, where the vulcanizable composition includes a filler, an oil and a curative.

51. The tire component of claim 50, where the filler includes silica derived from rice husk ash.

52. The tire component of claim 50, where the oil includes a bio-oil or a plant-based oil.
